(19) United States
(12) Patent Application Publication
(10) Pub. No.: US 2025/0099015 A1
(43) Pub. Date: Mar. 27, 2025

(54) A BRAIN CONTROL INTERFACE SYSTEM FOR DETERMINING A BASELINE FOR DETECTING BRAIN ACTIVITY

(71) Applicant: SIGNIFY HOLDING B.V., EINDHOVEN (NL)

(72) Inventors: ABHISHEK MURTHY, ARLINGTON, MA (US); DAKSHA YADAV, BOSTON, MA (US); PETER DEIXLER, ARLINGTON, MA (US)

(21) Appl. No.: 18/729,911

(22) PCT Filed: Jan. 13, 2023
(86) PCT No.: PCT/EP2023/050765

§ 371 Date: Jul. 18, 2024

Related U.S. Application Data
(60) Provisional application No. 63/300,747, filed on Jan. 19, 2022.

(30) Foreign Application Priority Data
Jan. 28, 2022 (EP) 22153802.8

(51) Int. Cl. A61B 5/378; A61B 5/00; A61B 5/256

(52) U.S. Cl. CPC A61B 5/378; A61B 5/256; A61B 5/7203

(57) ABSTRACT

A brain control interface system for determining a baseline for detecting brain activity of a user is disclosed. The brain control interface system comprising: a brain control interface configured to detect brain signals indicative of brain activity of a user in an environment, a memory configured to store activities of the user associated with different light scenes, a processor configured to: select, from the activities stored in the memory, a first activity of the user, control one or more lighting devices according to a first light scene associated with the first activity, detect brain signals of the user while the first light scene is active, determine, based on the detected brain signals, a first baseline for the brain signals, and store an association between the first baseline and the first light scene and/or the first activity.

### FIELD OF THE INVENTION

The invention relates to a brain control interface system for determining a baseline for detecting brain activity of a user. The invention further relates to a method of determining a baseline for detecting brain activity of a user, and to a computer program product for executing the method.

### BACKGROUND

Brainwave-based device control is a rising new technology. A brain-computer- interface (BCI) is used to detect brain signals of a user, whereupon information from these brain signals is derived. This information may, for example, be indicative of a thought, a mental state (e.g. happy, relaxed, concentrated, neutral) or an action of the user. The thought may, for example, be indicative of a control command for a controllable device, such as a lighting device. An example of such as system is disclosed in US10551921B2. There are two main types of BCIs: non-invasive and invasive BCIs. The non-invasive versions are the most common, and comprise sensors (electrodes) placed on the human head. These measure brain activity and translate that data to a computer. Most BCIs utilize electroencephalography (EEG) systems, which typically feature electrodes are attached to the scalp, which measure the electrical current sent by the neurons inside the brain. Changes in this electrical current reflect brain activity, because when an individual performs an action or thinks about something, hundreds of thousands of neurons are fired. This generates the electrical current, which is large enough to be measured on the scalp. A computer system then tries to make sense of this data to derive the user's action or thought. Alternatives to EEG systems are electrooculography (EOG), electromyography (EMG), electrodermal activity (EDA) and photoplethysmography (PPG) systems. As alternative to utilizing electrodes on the surface of the scalp, implantable brain-computer interfaces may be used. Here, probes are inserted into the brain through an automated process performed by a surgical robot. Each probe comprises an area of wires that contains electrodes capable of locating electrical signals in the brain, and a sensory area where the wire interacts with an electronic system that allows amplification and acquisition of brain signals.

Over the past several decades, neuroscientists have tried to decode the functionalities of different brain regions. For instance, it has been established that the occipital lobe is responsible for visual processing while the prefrontal cortex is responsible for problem-solving. Therefore, while a person is performing different activities during the day, different brain regions will be activated based on the skill that is required for a specific activity. For instance, when an individual passively views a presentation, the occipital lobe will be more involved as compared when a test is to be performed, which requires active problem-solving, and where the prefrontal cortex is used.

A research study (Min et al.—Bright Illumination Reduces parietal EEG alpha activity during a sustained attention task. Brain Research, 2013.) conducted several experiments of sustained attention on subjects under different illumination conditions. EEG was recorded from the parietal region of the brain. The study found that brain pulses were significantly influenced by the illuminance factor. Their mean values indicate that high illuminance resulted in significantly longer latencies than low illuminance. The study concluded that the illumination condition substantially influences the attentional processing as reflected in the significant modulations of EEG activity.

A related study (Figueiro et al.—Preliminary evidence that both blue and red light can induce alertness at night. BMC Neuroscience 2009; 10:105-105) shows that both short-wavelength and long-wavelength light increase alertness at night, as shown in EEG power change. Additionally, 10 lx of red light is also found to significantly affect the EEG measures compared to preceding dark conditions. In another study (Plitnick et al—The effects of red and blue light on alertness and mood at night. Lighting Research and Technology 2010; 42:449-458), two levels (10 lx and 40 lx) of blue and red lights were both found to increase EEG beta power.

In a related study (Lin, Jing et al—Effect of long-wavelength light on electroencephalogram and subjective alertness, Lighting Research and Technology, 2020 Jan. 5, Vol. 52) it was investigated how exposures to long-wavelength lights of two different levels (40 lx and 160 lx) affect objective alertness (as measured by EEG). A significant effect of light levels on EEG beta (13-30 Hz) power was observed. Exposure to both 40 lx and 160 lx long-wavelength lights significantly increased beta power compared to the Dim condition.

In a related study (Ackeren et al.—A (blue) light in the dark: Blue light modulates oscillatory alpha activity in the occipital cortex of totally visually blind individuals with intact non-visual photoreception), three participants who were visually blind but had intact non-visual responses, were subjected to an on-off patterns of blue light. The study concluded that the blue light impacts the occipital region of the brain and decreases the power of the alpha EEG rhythm in this specific part of the brain.

Since EEG is a time-resolving signal, it may often have temporal drifts which are unrelated to the detection tasks of the BCI (e.g. detecting a change in user's emotion state or the user issuing a brain command). Various internal and external sources may cause temporal drifts, which change over time, and also across electrodes. To reduce the effect of such drifts, it is custom to perform a so-called baseline correction. Essentially, this uses EEG activity over a baseline period, i.e. before an external event occurs, to correct activity over a post-stimulus interval, i.e. the time after an external event occurs. Various approaches exist for baseline correction. The traditional way is subtracting the mean of a baseline period from every time point of the baseline and post-stimulus interval. In other words, the average voltage values of each electrode are calculated within a time interval and then this average is subtracted from that time interval of the signal.

WO 2012044261 A1 discloses that baseline calibration operations can include eyes open calibration operations in which eyes open baseline EEG data is captured while the user directs their field of view to a portion of their surrounding environment that lacks visual stimuli corresponding to or correlated with the visual stimulus generators' presentation frequencies. In certain embodiments, eyes open baseline EEG data can be captured while the user looks at a neutral baseline scene such as a wall having a uniform of substantially uniform colour scheme, and which is uniformly or substantially uniformly illuminated by a wide spectrum optical signal source (e.g., a white wall illuminated with ordinary room lighting).

### SUMMARY OF THE INVENTION

The inventors have realized that while a person is performing different activities during the day, different brain regions will be activated based on the skill that is required for a specific activity. Additionally, the illumination of the space wherein the user performs a specific activity affects the brain signals of the user. When brain signals are analyzed, for instance by a head-mounted consumer brain-computer-interface (BCI), a baseline is typically used. The baseline can be considered as reference brain signals that are present when there's low/minimal brain activity of the user, for instance when the user is not providing a brain-control command to switch on the light. Alternatively, the baseline can be considered as reference brain signals that are present when there's constant brain activity of the user, for instance when the user is reading a book or meditating. The BCI may effectively analyze the delta between the measured brain signals and the baseline to for instance deduce whether the user has issued the BCI command for switching on a ceiling lighting unit. The inventors have realized that, since the environmental illumination and the user activity both affect the brain signals of a user (and thereby the detection requirements for properly detecting the brain signals), it is beneficial to set a specific baseline for a specific user activity and/or specific light settings, because this improves the brain signal detection. It is therefore an object of the present invention to improve the robustness of the brain signal detection performed by an BCI.

According to a first aspect, the object is achieved by a brain control interface system for determining a baseline for detecting brain activity of a user, the brain control interface system comprising:
a brain control interface configured to detect brain signals indicative of brain activity of a user in an environment,
a memory configured to store activities of the user associated with different light scenes,
a processor configured to:
   select, from the activities stored in the memory, a first activity of the user,
   control one or more lighting devices according to a first light scene associated with the first activity,
   detect brain signals of the user while the first light scene is active,
   determine, based on the detected brain signals, a first baseline for the brain signals, and
   store an association between the first baseline and the first light scene and/or the first activity.

When a user performs a specific activity, the one or more lighting devices may be controlled according to a light scene that corresponds with that specific user activity. Alternatively, the one or more lighting devices may be purposefully controlled to improve brain signal detection during a specific activity, for instance emotion state detection. The brain control interface system is configured for setting baselines for brain signals under different lighting conditions for specific activities. The memory stores activities of the user associated with different light scenes. The processor selects an activity from the memory and controls the one or more lighting devices located in the user's environment according to a light scene associated with that activity. The processor then analyzes brain signals (as detected by the brain control interface) while the first light scene is active in the user's environment and determines (establishes) a baseline for the brain signals (i.e. while the user is exposed to the light scene). The processor then stores an association between the first baseline and the light scene and/or the first activity, such that the baseline can be applied for brain signal analysis when the user is performing the activity and/or when the light scene is active. The association may be stored in the memory, or in another memory. Configuring the brain control interface system by setting a baseline for a specific activity and/or lighting scene is beneficial, because the baseline can be applied for brain signal analysis when the user is performing the activity and/or when the light scene is active. This is advantageous, because by setting a dedicated baseline for the activity and/or the light scene, brain signal detection is improved as for instance the influence of different illuminance factors on the brain pulses are significantly reduced.

The brain control interface system may be configured to determine different baselines for different activities and corresponding light scenes. The processor may be further configured to
select, from the activities stored in the memory, a second activity of the user,
control the one or more lighting devices according to a second light scene associated with the second activity,
detect brain signals while the second light scene is active,
determine, based on the detected brain signals, the second baseline for the user for the second activity, and
store a second association between the second baseline and the second light scene and/or the second activity. In other words, the processor may repeat the steps for a second activity with a corresponding second light scene.

The first activity may be an activity that requires detection of brain signals from a first region of the brain of the user, and the second activity may be an activity that requires detection of brain signals from a second, different, region of the brain of the user. The functions of each brain lobe of the human brain and their relation to activities such as reading, listening to music, executive planning, positive emotions, problem solving, motor skills, etc. are well known in the art, for instance from neurofeedback training. The first light scene may be a light scene with a spectrum and/or intermittent photic stimulation that affects the first region of the brain, and the second light scene may be a light scene with a spectrum and/or intermittent photic stimulation that affects the second region of the brain. The first light scene may be a light scene with a spectrum t and/or intermittent photic stimulation hat positively affects the first region of the brain such that the intensity of the brain signals in the first region is reduced. Similarly, the second light scene may be a light scene with a spectrum and/or intermittent photic stimulation that positively affects the second region of the brain such that the intensity of the brain signals in the second region is reduced. Different user activities may require analysis for different brain regions. For instance, when a user views a presentation, their occipital lobe will be more involved as compared when the user is performing a test, which requires problem-solving. In that case, prefrontal cortex will be more utilized. Hence, it is beneficial to provide different baselines for different brain regions.

The first and second brain regions may be one of: the occipital region of the brain, the parietal region of the brain, the prefrontal cortex and the temporal lobe. Certain light effects impact certain regions of the brain. For instance, blue light impacts the occipital region of the brain and decreases the power of the alpha EEG rhythm in this part of the brain. In another example, high illuminance results in significantly longer latencies than low illuminance for the parietal region of the brain.

The first light scene may be a light scene with a spectrum and/or intermittent photic stimulation that positively affects brain signals of a brain region of the brain, the brain region being associated with the first activity. The memory may be further configured to store this association. Hence, the memory may store a correlation between the activity,

The processor may be configured to obtain data indicative of that the first light scene has been activated, to retrieve the stored first baseline associated with the first light scene, and (while the first light scene is active) to apply the stored first baseline to brain signals while the first light scene is active. Additionally or alternatively, the processor may be configured to obtain data indicative of that the user is performing the first activity, to retrieve the stored first baseline, and to apply the stored first baseline to brain signals while the user is performing the first activity. The processor may be configured to switch between a configuration mode and an operation mode. The processor may be configured to perform the steps related to determining and storing the (first) baseline when it is set to the configuration mode. The processor may be configured to perform the steps related to obtaining the data indicative of that the first light scene has been activated and/or the data indicative of that the user is performing the first activity when the processor is set to the operation mode. In other words, the processor may first determine the baselines in the configuration mode, and then apply the baselines in operation mode. The processor may apply the baselines in operation mode to reduce the effect of lighting/activity-induced temporal drifts in the acquired EEG data which are unrelated to the detection tasks of the BCI of detecting a change in user's emotion state or the user issuing a brainwave command. For instance, the baseline correction may subtract in operation mode the mean of a baseline period (e.g. average voltage value of each electrode) from the voltage value of every time point.

The processor may be further configured to obtain data indicative of that the user is performing the second activity, to retrieve the stored second baseline, and to apply the stored second baseline to brain signals while the user is performing the second activity.

The first activity may be an expected activity that the user will perform during the day. The processor may be configured to apply a respective baseline for the expected activity before (and while) the user performs the respective expected activity.

The processor may be further configured to determine the expected activity by accessing a user schedule comprising data indicative of scheduled activities. Alternatively, the processor may be further configured to determine the expected activity based on historical activities of the user or historical activities of other users.

The processor may be further configured to analyze brain signals while the first light scene is active to identify a level of noise in the brain signals, and, if the level of noise exceeds a first threshold, select a different light scene from the memory or adjust the first light scene (light spectrum and/or light intensity) until a target level of noise in the brain signals has been established. By adjusting the light scene of the one or more lighting devices (and therewith the light output of the one or more lighting devices), the effect of the light output on the level of noise in the brain signals is reduced. Since the light effects provided by the one or more lighting devices affect the brain signals, it is beneficial to adjust the light scene to reduce the effect of the light effects. By adjusting the light scene, the brain control interface system reduces the chance of false/incorrect triggers. The adjusting of the of light scene may be incrementally adjusting the lighting scene.

According to a second aspect, the object is achieved by a method of determining a baseline for detecting brain activity of a user, the method comprising:
selecting, from activities stored in a memory, the activities of the user being associated with different light scenes, a first activity of the user,
controlling one or more lighting devices according to a first light scene associated with the first activity,
analyzing brain signals, detected by a brain control interface, of the user while the first light scene is active,
determining, based on the detected brain signals, a first baseline for the brain signals, and
storing an association between the first baseline and the light scene and/or the first activity.

According to a third aspect, the object is achieved by a computer program product for a computing device, the computer program product comprising computer program code to perform the method when the computer program product is run on a processing unit of the computing device.

It should be understood that the method and the computer program product may have similar and/or identical embodiments and advantages as the above-mentioned systems.

### BRIEF DESCRIPTION OF THE DRAWINGS

The above, as well as additional objects, features and advantages of the disclosed systems, devices and methods will be better understood through the following illustrative and non-limiting detailed description of embodiments of devices and methods, with reference to the appended drawings, in which:

FIG. 1 shows schematically an example of a brain control interface system;

FIG. 2a shows schematically an example of a baseline of a set of detected brain signals;

FIG. 2b shows schematically an example of a set of brain signals relative to a baseline threshold; and

FIG. 3 shows schematically an example of a method of determining a baseline for detecting brain activity of a user.

All the figures are schematic, not necessarily to scale, and generally only show parts which are necessary in order to elucidate the invention, wherein other parts may be omitted or merely suggested.

### DETAILED DESCRIPTION

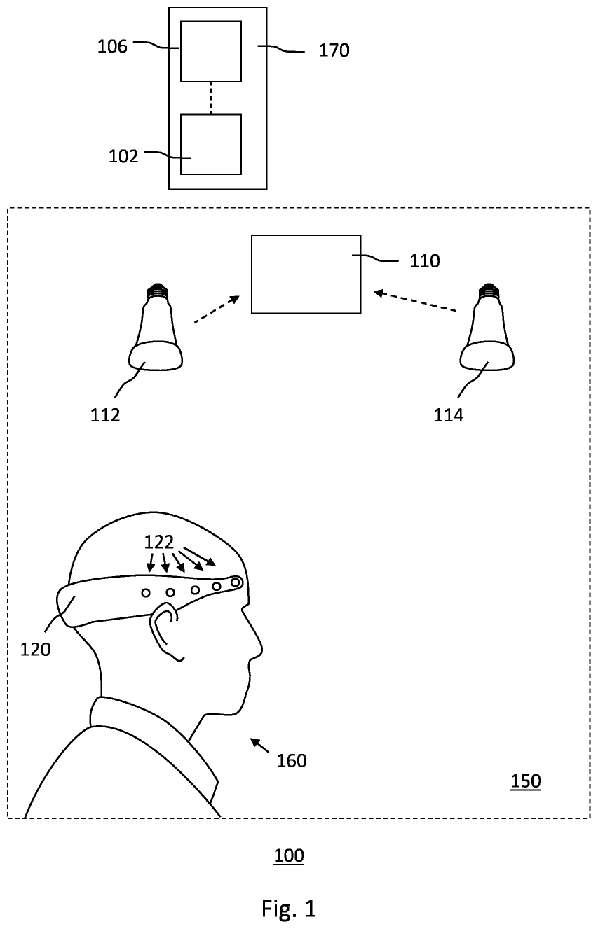

FIG. 1 shows schematically an overview of a brain control interface system 100. The brain control interface system 100 comprises a brain control interface 120 (e.g. a head-worn device). The brain control interface 120 (BCI) is configured to detect brain signals indicative of brain activity of a user 160 in an environment 150. The system 100 further comprises or more processors 106 configured to analyze the brain signals. The BCI 120 may comprise one or more electrodes 122 in contact with the user's scalp, which electrodes 122 are used for detecting EEG signals of the user. It should be understood that such a BCI 120 is an example, and that other types of brain signal detection may be used.

The brain control interface system 100 further comprises one or more processors 106 (e.g. circuitry, one or more microcontrollers, etc.). The one or more processors 106 are configured to obtain data indicative of the brain signals as detected by the BCI 120. The one or more processors 106 may be comprised in a single device or distributed across multiple devices, which may depend on the system architecture of the BCI system 100. For instance, in the example of FIG. 1, the one or more processors 106 and the memory 102 are comprised in a single device 170, which device 170 is communicatively coupled with the lighting controller 110 and the BCI 120. It should be understood that this system architecture is merely an example, and that the skilled person is able to design alternative system architectures without departing from the scope of the appended claims. For instance, a first processor of the one or more processors 106 may be comprised in the BCI 120, and a second processor on a remote server or in the lighting controller 110. In another example, the one or more processors 106 and the memory 102 may be comprised in the lighting controller 110, or in the BCI. In another example, a first processor of the one or more processors 106 may be comprised in a remote server and a second processor in the lighting controller 110.

The brain control interface system 100 further comprises a memory 102 configured to store activities of the user 160 associated with different light scenes. The processor is configured to determine a baseline for the brain activity detection of the user 160. The processor 106 is configured to select, from the activities stored in the memory 102, a first activity of the user 160. The memory 102 may store a list of user activities, each associated with a light scene (i.e. one or more light settings for the one or more lighting devices 112, 114. The activities may be defined as (daily) tasks the user 160 performs (studying, working, performing a test, relaxing, etc.). Alternatively, the activities may be defined as required cognitive abilities of the user 160. Depending on the user's activity a specific cognitive ability may be required. Examples of cognitive abilities include perception (ability to recognize and interpretate of sensory stimuli), attention (ability to sustain concentration), memory (ability to store inputs), motor skills (ability to mobilize), visual and special processing (ability to process incoming visual stimuli) and executive abilities like problem-solving, pattern recognition, inhibition, etc. One or more activities/required abilities may be associated with a light scene. The processor 106 may then determine a baseline for a specific activity by selecting that activity from the memory 102, and control the one or more lighting devices 112, 114 according to a light scene associated with that activity. This process may be initiated by the user 160 (who wishes to configure the brain control interface system 100), for instance via a user interface of the BCI, or via another user interface (e.g. via a user interface of a mobile phone, a (tablet) pc, etc.). Alternatively, the process may be initiated automatically by the processor 106, for instance when the brain control interface system 100 is used for the first time, when the brain control interface system 100 requires reconfiguration/recalibration, when a new user is identified, etc.

The processor 106 may be configured to control the one or more lighting devices 112, 114 directly, or via a lighting controller 110. The processor 106 may be comprised in a single device 170, which device 170 may be communicatively coupled with the one or more lighting devices 112, 114, for instance directly or via the lighting controller 110. The processor 106 may be configured to control the one or more lighting devices 112, 114 by communicating lighting control signals to the one or more lighting devices 112, 114 to generate a light scene. The processor 106 may control the one or more lighting devices 112, 114 via a communication unit (not shown) configured to communicate with the lighting devices via a wireless network, such as a Zigbee, BLE, Wi-Fi or Thread network). In embodiments, the processor 106 may be located in the lighting controller 110 or in a lighting device, the processor 106 may communicate control signals to the one or more lighting devices 112, 114 directly. The control signals comprise light settings indicative of light output properties (for example hue, saturation, brightness, beam direction, etc.). The one or more lighting devices 112, 114 are configured to receive the control signals and a driver is configured to adjust the light output of one or more (LED) light sources accordingly.

After controlling the one or more lighting devices 112, 114 in the environment 150 according to the first light scene associated with the first activity, the processor 106 detects brain signals of the user 160 in the environment 150 while the first light scene is active (and the user is not performing the first activity). The processor 106 may analyze these brain signals and determine, based on the detected brain signals, a first baseline for the brain signals (and therewith a baseline for the activity and the light scene). A baseline can be considered as reference brain signals, used as a reference for detecting additional brain signals while the user is performing activities. The (first) baseline is captured under specific lighting conditions associated with a specific activity. This baseline may be used when the user 160 is performing the specific activity and/or when the light scene is active in the environment 150. The processor 106 is configured to store an association between the first baseline and the first light scene and/or the first activity. The association may be stored in the memory 102, in a memory of the brain control interface 120, in a remote memory accessible via a network (e.g. the internet), etc. By storing the determined first baseline for the first activity and the first light scene (e.g. in an configuration mode), it is obtainable for later use (e.g. in an operation mode).

The processor 106 may be further configured to repeat this process for further activities and associated light scenes. The processor 106 may be configured to select, from the activities stored in the memory 102, a second activity of the (same) user 160, control the one or more lighting devices in the environment 150 according to a second light scene associated with the second activity, detect brain signals while the second light scene is active, determine, based on the detected brain signals, the second baseline for the user 160 for the second activity, and store a second association between the second baseline and the second light scene and/or the second activity.

The first activity may be an activity that requires detection of brain signals from a first region of the brain of the user 160, and the second activity may be an activity that requires detection of brain signals from a second, different, region of the brain of the user 160. The first light scene may be a light scene with a spectrum and/or intermittent photic stimulation that affects the first region of the brain, and the second light scene may be a light scene with a spectrum and/or intermittent photic stimulation that affects the second region of the brain. The first light scene may be a light scene with a spectrum and/or intermittent photic stimulation that positively affects the first region of the brain such that the intensity of the brain signals in the first region is reduced (and thereby subsequently improving for instance the signal to noise ratio of the additional brain signal measurement data associated with the user performing certain activity(ies)). Similarly, the second light scene may be a light scene with a spectrum and/or intermittent photic stimulation that positively affects the second region of the brain such that the intensity of the brain signals in the second region is reduced. Different user activities may require analysis for different brain regions. For instance, when a user views a presentation, their occipital lobe will be more involved as compared when the user is performing a test in school, which requires problem-solving. In that case, prefrontal cortex will be more utilized. The first and second brain regions may be one of: the occipital region of the brain, the parietal region of the brain, the prefrontal cortex and the temporal lobe. Certain light effects impact certain regions of the brain. For instance, blue light impacts the occipital region of the brain and decreases the power of the alpha EEG rhythm in this part of the brain. In another example, high illuminance results in significantly longer latencies than low illuminance for the parietal region of the brain.

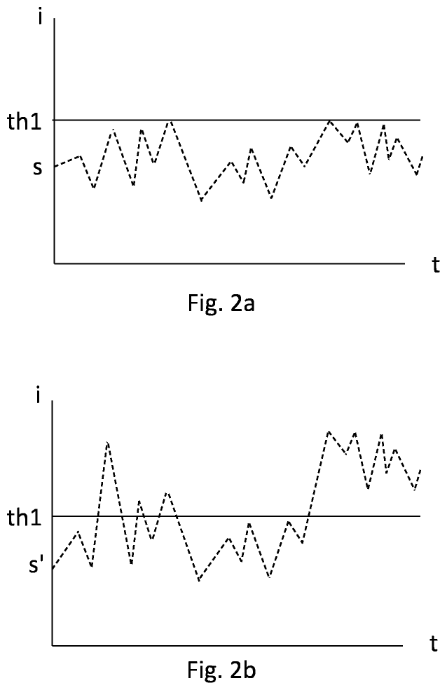

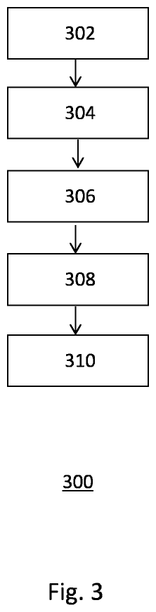

FIG. 2a shows a graph schematically illustrating brain signals s with intensity i captured over time t, and a threshold value th. The brain signals s have been detected while a specific light scene (associated with a specific activity) is active. The processor 106 may determine, based on the detected brain signals s, a baseline for the brain signals. The baseline may be defined as the actual signals s as reference brain signals (which may be used as a reference for detecting brain signals while the user is performing the activity). Additionally or alternatively, the baseline may be defined as a threshold value th (which may be used as a reference for detecting brain signals while the user is performing the activity). FIG. 2b shows a graph schematically illustrating brain signals s′ with intensity i captured over time t, and the threshold value th of FIG. 2a, which brain signals s′ are captured while the user is performing the activity and/or when the light scene is active. The baseline of FIG. 2a may be applied as a reference baseline, or the threshold th may be applied as a reference for analyzing the brain signals s. If the brain signals s′ sufficiently deviate from the reference baseline or the threshold th, the brain activity during the performing of the activity may be determined. This brain activity may be determined by analyzing the brain signals, and be used (e.g. by the processor 106) to, for example, derive information about the user, for instance about the mental/emotional state of the user. Additionally or alternatively, the brain activity may be determined by analyzing the brain signals, and be used (e.g. by the processor 106) to, for example, derive a specific control command for controlling a controllable device (such as a lighting device) from the brain signals.

The processor 106 may be configured to be set to a configuration mode. When set to the configuration mode, the processor 106 may determine the baseline for the one or more activities and associated light scenes (i.e. the steps of selecting, from the activities stored in the memory 102, an activity of the user 160, controlling one or more lighting devices 112, 114 according to a light scene associated with the activity, detecting brain signals of the user while the light scene is active, determining, based on the detected brain signals, a baseline for the brain signals, and storing an association between the baseline and the light scene and/or the activity). The processor 106 may be configured to switch from the configuration mode to an operation mode, wherein, when set to the operation mode, the processor 106 is configured to apply the determined baseline to brain signals received from the BCI 120 when the user is performing the respective activity and/or when the respective light scene is active.

The processor 106 may be configured to switch between the configuration mode and the operation mode upon receiving a switching signal. The switching signal may be received a user interface, wherein a user provides an input via the user interface to switch from the configuration mode to the control mode. Alternatively, the switching signal may be generated by the processor 106 if one or more conditions are fulfilled (e.g. if a predefined number of baselines has been set, if baselines have been set for all activities and their respective light scenes, etc.). Alternatively, the switching signal may be received from a remote device, which instructs the processor 106 to switch to the operation mode.

The processor 106 may be configured to obtain data indicative of that the first light scene has been activated, to retrieve the stored first baseline associated with the first light scene, and to apply the stored first baseline to brain signals while the first light scene is active. The processor 106 may, for example, receive one or more signals (e.g. from the lighting controller 110, from the one or more lighting devices 112, 114, from a remote control system, etc.) indicative of that a specific light scene has been activated, and apply the baseline (correction) to the detected brain signals. The processor 106 may be configured to obtain data indicative of that the second light scene has been activated, to retrieve the stored second baseline associated with the second light scene, and to apply the stored second baseline to brain signals while the second light scene is active.

Additionally or alternatively, the processor 106 may be configured to obtain data indicative of that the user is performing the first activity, to retrieve the stored first baseline, and to apply the stored first baseline to brain signals while the user is performing the first activity. The processor 106 may, for example, receive one or more signals (e.g. from a remote control system, from the BCI 120, from a user device (e.g. a smartphone or a smartwatch), etc.) indicative of that the user is performing a specific activity, and apply the baseline to the detected brain signals when the user is performing the activity. Sensors for detecting activities of users, or systems for determining activities of users, are known in the art and will therefore not be discussed in detail. The processor 106 may be further configured to obtain data indicative of that the user 160 is performing the second activity, to retrieve the stored second baseline, and to apply the stored second baseline to brain signals while the user is performing the second activity.

The first activity may be an expected activity that the user will perform during the day. The processor 106 may be configured to apply a respective baseline for the expected activity before (and while) the user performs the respective expected activity.

The processor may be further configured to determine the expected activity by accessing a user schedule comprising data indicative of scheduled activities. The schedule may be stored on a memory, for instance in a remote (cloud) memory or in a memory of a user device. Alternatively, the processor 106 may be further configured to determine the expected activity based on historical activities of the user or historical activities of other users. The processor 106 (or a further processing device) may be configured to monitor activities of the user 160 over time, analyze these activities and predict when the user will perform a specific activity. Techniques for determining expected activities of a user are known in the art and will therefore not be discussed in detail.

The processor 106 may be further configured to analyze brain signals while the first light scene is active to identify a level of noise in the brain signals, and, if the level of noise exceeds a first threshold, select a different light scene from the memory or adjust the first light scene until a target level of noise in the brain signals has been established. Light impacting the brain signals may originate from artificial lighting and/or natural daylight. The natural daylight present in the room may depend on the time of the day and/or the current position of window blinds. The processor 106 may, for example, compare the detected brain signals to reference brain signals to determine the level of noise based on the differences between the detected brain signals and the reference brain signals. Additionally or alternatively, the processor 106 may compare the detected brain signals with one or more thresholds and/or baselines to determine level of noise.

For instance, dynamic effects, and more specifically dynamic effects with higher dynamics levels, for instance an entertainment light effect with multiple light changes per second, may affect brain signals originating from certain regions of the brain, and the one or more processors 106 may be configured to analyze these regions for noise to determine the level of noise. It is known in the art that EEG measurement data may react to dynamic stimulation with light. For instance, photosensitivity is a condition which can be detected on EEG data as a paroxysmal reaction to Intermittent Photic Stimulation (IPS). This EEG response, elicited by IPS or by other visual stimuli of daily life, is called Photo Paroxysmal Response (PPR). PPRs are well documented in both epileptic and non-epileptic subjects. Photic Stimulation (PS) is in the medical setting used as a standard procedure during routine EEG recording. The procedure is typically carried out with strobe units that flash for 5-10 seconds at frequencies typically between 1 and 35 Hz (see: Lara V Marcuse MD et al, in Rowan's Primer of EEG (Second Edition), 2016). PS evokes a rhythmic frequency in the occipital derivations termed photic driving.

Another example of dynamic lighting affecting the measured EEG signals, is the photomyogenic EEG response (see: Human photosensitivity: from pathophysiology to treatment, Verrotti A et al., Eur J Neurol. 2005 November; 12(11):828-41). The photomyogenic EEG response is observed in non-epileptic subjects resulting from stimulus time-locked rhythmic contractions of periocular and facial muscles in phase with the flash stimulus frequency, which can extend to myoclonic jerking of the muscles of the upper body. The photomyogenic response in the EEG signals ceases directly after flash stimuli are stopped.

Certain colors of light may also affect the brain signals. For instance, short wavelength light, such as blue light, impacts the occipital region of the brain and decreases the power of the alpha EEG rhythm in this specific part of the brain. Similarly, long wavelength light, such as red light, affects the beta EEG rhythm. The processor 106 may be configured to analyze a specific region associated with that light scene for noise to determine the level of noise.

FIG. 4 shows schematically a method 300 of determining a baseline for detecting brain activity of a user, the method comprising: selecting 302, from activities stored in a memory, the activities of the user being associated with different light scenes, a first activity of the user, controlling 304 one or more lighting devices according to a first light scene associated with the first activity, analyzing 306 brain signals, as detected by a brain control interface, of the user while the first light scene is active, determining 308, based on the detected brain signals, a first baseline for the brain signals, and storing 310 an association between the first baseline and the light scene and/or the first activity.

The method 300 may be executed by computer program code of a computer program product when the computer program product is run on a processing unit of a computing device, such as the processor 106 of the system 100.

It should be noted that the above-mentioned embodiments illustrate rather than limit the invention, and that those skilled in the art will be able to design many alternative embodiments without departing from the scope of the appended claims.

In the claims, any reference signs placed between parentheses shall not be construed as limiting the claim. Use of the verb “comprise” and its conjugations does not exclude the presence of elements or steps other than those stated in a claim. The article “a” or “an” preceding an element does not exclude the presence of a plurality of such elements. The invention may be implemented by means of hardware comprising several distinct elements, and by means of a suitably programmed computer or processing unit. In the device claim enumerating several means, several of these means may be embodied by one and the same item of hardware. The mere fact that certain measures are recited in mutually different dependent claims does not indicate that a combination of these measures cannot be used to advantage.

Aspects of the invention may be implemented in a computer program product, which may be a collection of computer program instructions stored on a computer readable storage device which may be executed by a computer. The instructions of the present invention may be in any interpretable or executable code mechanism, including but not limited to scripts, interpretable programs, dynamic link libraries (DLLs) or Java classes. The instructions can be provided as complete executable programs, partial executable programs, as modifications to existing programs (e.g. updates) or extensions for existing programs (e.g. plugins). Moreover, parts of the processing of the present invention may be distributed over multiple computers or processors or even the ‘cloud’.

Storage media suitable for storing computer program instructions include all forms of nonvolatile memory, including but not limited to EPROM, EEPROM and flash memory devices, magnetic disks such as the internal and external hard disk drives, removable disks and CD-ROM disks. The computer program product may be distributed on such a storage medium, or may be offered for download through HTTP, FTP, email or through a server connected to a network such as the Internet.

1. A brain control interface system for determining a baseline for detecting brain activity of a user, the brain control interface system comprising:
a brain control interface configured to detect brain signals indicative of brain activity of a user in an environment,
a memory configured to store activities of the user associated with different light scenes,
a processor configured to:
select, from the activities stored in the memory, a first activity of the user,
control one or more lighting devices according to a first light scene associated with the first activity,
detect brain signals of the user while the first light scene is active,
determine, based on the detected brain signals, a first baseline for the brain signals, and
store an association between the first baseline and the first light scene and/or the first activity,
wherein the processor is further configured to:
select, from the activities stored in the memory, a second activity of the user,
control the one or more lighting devices according to a second light scene associated with the second activity,
detect brain signals while the second light scene is active,
determine, based on the detected brain signals, the second baseline for the user for the second activity, and
store a second association between the second baseline and the second light scene and/or the second activity.

2. The brain control interface system of claim 1, wherein the first activity is an activity that requires detection of brain signals from a first region of the brain of the user, and wherein the second activity is an activity that requires detection of brain signals from a second, different, region of the brain of the user, and wherein the first light scene is a light scene with a spectrum and/or intermittent photic stimulation that affects the first region of the brain, and wherein the second light scene is a light scene with a spectrum and/or intermittent photic stimulation that affects the second region of the brain.

3. The brain control interface system of claim 2, wherein the first and second brain region are one of: the occipital region of the brain, the parietal region of the brain, the prefrontal cortex and the temporal lobe.

4. The brain control interface system of claim 1, wherein the first light scene is a light scene with a spectrum and/or intermittent photic stimulation that positively affects brain signals of a brain region of the brain, the brain region being associated with the first activity.

5. The brain control interface system of claim 1 wherein the processor is configured to obtain data indicative of that the first light scene has been activated on the one or more lighting devices, to retrieve the stored first baseline associated with the first light scene, and to apply the stored first baseline to brain signals while the first light scene is active.

6. The brain control interface system of claim 1, wherein the processor is configured to obtain data indicative of that the user is performing the first activity, to retrieve the stored first baseline associated with the first activity, and to apply the stored first baseline to brain signals while the user is performing the first activity.

7. The brain control interface system of claim 6, wherein the processor is further configured to obtain data indicative of that the user is performing the second activity, to retrieve the stored second baseline, and to apply the stored second baseline to brain signals while the user is performing the second activity.

8. The brain control interface system of claim 6, wherein the processor is further configured to control the one or more lighting devices according to the first light scene while the user is performing the first activity.

9. The brain control interface system of claim 1, wherein the first activity is an expected activity that the user will perform during the day.

10. The brain control interface system of claim 9, wherein the processor is further configured to determine the expected activity by accessing a user schedule comprising data indicative of scheduled activities.

11. The brain control interface system of claim 9, wherein the processor is further configured to determine the expected activity based on historical activities of the user or historical activities of other users.

12. The brain control interface system of claim 1, wherein the processor is further configured to analyze brain signals while the first light scene is active to identify a level of noise in the brain signals, and, if the level of noise exceeds a first threshold, select a different light scene from the memory or adjust the first light scene until a target level of noise in the brain signals has been established.

13. A method of determining a baseline for detecting brain activity of a user, the method comprising:
selecting, from activities stored in a memory, the activities of the user being associated with different light scenes, a first activity of the user,
controlling one or more lighting devices according to a first light scene associated with the first activity,
analyzing brain signals, detected by a brain control interface, of the user while the first light scene is active,
determining, based on the detected brain signals, a first baseline for the brain signals, and
storing an association between the first baseline and the light scene and/or the first activity,
wherein the method further comprises:
selecting, from the activities stored in the memory, a second activity of the user,
controlling the one or more lighting devices according to a second light scene associated with the second activity,
detecting brain signals while the second light scene is active,
determining, based on the detected brain signals, the second baseline for the user for the second activity, and
storing a second association between the second baseline and the second light scene and/or the second activity.

14. A computer program product for a computing device, the computer program product comprising computer program code to perform the method of claim 13 when the computer program product is run on a processing unit of the computing device.
